(19) United States
(12) Patent Application Publication
(10) Pub. No.: US 2009/0118824 A1
(43) Pub. Date: May 7, 2009

(54) Heart Valve Prosthesis

(76) Inventor: Alexander Samkov, Moscow (RU)

Correspondence Address: Aleksandr Smushkovich, POB 140505, Brooklyn, NY, 11214, US

(21) Appl. No.: 12/225,973

(22) PCT Filed: Apr. 28, 2006
(86) PCT No.: PCT/IB2006/001066

§ 371 Date: Oct. 3, 2008

(30) Foreign Application Priority Data
Apr. 4, 2006 (RU) RU2006110832

(51) Int. Cl. A61F 2/24

(52) U.S. Cl. 623/2.2; 623/2.38

(57) ABSTRACT

A heart valve prosthesis includes an annulus and a closing element having three leaflets. Hinge mechanisms are provided for attaching each leaflet to a top region of an interior surface of the annulus. In one example, the leaflets have convexo-concave surfaces and a non-uniform thickness. In another example, the top region of the interior surface has two sets of ledges which can be formed by intersecting three pairs of cylindrical surfaces. Ledges within each set are evenly spaced about the interior surface, a ledge in one set alternating with a ledge in the other set. In a further example, a bottom region of the interior surface is formed by intersecting three cylindrical surfaces.

### RELATED APPLICATIONS

This application claims the benefit of Russian Patent Application No. 2006110832, filed on Apr. 4, 2006, the contents of which are incorporated herein by reference in their entirety.

### BACKGROUND OF THE INVENTION

One type of artificial heart valves is the mechanical heart valve, generally manufactured from artificial materials such as titanium, titanium alloys, graphite and others.

In a common design, mechanical heart valves include two leaflets held in a casing by hinges. When open, the blood flow through a bi-leaflet valve forms a central stream, between the two leaflets and two side streams. Deficiencies associated with bi-leaflet designs include turbulence, velocity gradients, non-synchronous opening of the leaflets and recirculation of the blood resulting in an increased burden on the heart.

Tri-leaflets designs also have been developed. Known in the art, for instance, is a heart valve having flat leaflets as disclosed in U.S. Pat. No. 5,207,707, issued to Gourley on May 4, 1993. Flat leaflets also are used in the heart valve disclosed in Russian Patent document RU 2,173,969, published in 2001.

U.S. Pat. No. 6,569,197 B2, issued to Samkov et al. on My 27, 2003, discloses an artificial heart valve prosthesis having an annular body which includes two flanges of different thickness and which houses three leaflets, each having a flat and a concave-spherical surface.

A tri-leaflet mechanical heart valve which includes three fan-shaped convexo-concave leaflets is disclosed in U.S. Patent Application Publication No. 2002/0022879 A1, published on Feb. 21, 2002, and U.S. Pat. No. 6,896,700, issued on May 24, 2005, both to Lu et al.

Both bi-leaflet and tri-leaflet mechanical heart valves often are associated with thrombosis and thromboembolism. Some tri-leaflet heart valve designs promote formation of stagnant zones in the blood flow, thus increasing the danger of blood clotting. In other cases, tri-leaflet valves continue to generate blood flow turbulence. Designs that place a high mechanical load on the hinge mechanisms result in an increased likelihood of early failure of the implant. Difficult machining-is-still another disadvantage associated with some of the existing tri-leaflet heart valves.

A need continues to exist therefore for improving mechanical heart valve prostheses. More specifically, a need continues to exist for tri-leaflet artificial heart valves that reduce or eliminate problems associated with existing designs.

### SUMMARY OF THE INVENTION

The invention generally relates to artificial heart valve prostheses, more specifically to a tri-leaflet artificial heart valve.

In one embodiment, a heart valve prosthesis includes three leaflets attached to an annulus, each leaflet having convexo-concave surfaces and a non-uniform thickness.

In another embodiment, a heart valve prosthesis, includes a closing element having three flaps and an annulus for housing the closing element, the annulus having an interior surface. At a top region, the interior surface includes two sets of ledges. Ledges within each set are evenly spaced about the interior surface, a ledge in one set alternating with a ledge in the other set.

In a further embodiment, a heart valve prosthesis includes a closing element having three flaps, an annulus for housing the closing element, the annulus having an annulus axis and an interior surface and three ledges formed at the interior surface by three intersecting cylindrical surfaces evenly disposed within the annulus, each cylindrical surface having a cylindrical axis displaced from the annulus axis. At each ledge there are two sockets for attaching the flaps to the interior surface.

The invention also is related to a leaflet shaped for a tri-leaflet artificial heart valve prosthesis. The leaflet has convexo-concave surfaces and a non-uniform thickness.

The invention has many advantages. For instance, the tri-leaflet artificial heart valve prosthesis disclosed herein resembles the human aortic valve and is expected to provide long and reliable service. The invention decreases or minimizes blood flow turbulence and reduces or eliminates formation of stagnant zones. It provides for a controlled reverse flow, thereby reducing or preventing blood clotting. In preferred embodiments, six cylindrical surfaces are used to shape the upper region of the annulus. Together with the three leaflets, the cylindrical surfaces give rise to essentially independent blood flow channels for washing or flushing the hinges that connect the leaves to the annulus, thus reducing the likelihood of thromboembolism. Other aspects of the invention promote good opening and closing conditions and minimize blood recirculation. Furthermore, the invention can reduce hydraulic resistance and improve the utilization of the valve cross sectional area. In specific aspects the invention also relates to an artificial heart valve prosthesis having an easy to manufacture casing.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a view on a diametrical plane of a heart valve prosthesis with flaps in the closed position.

FIG. 2 is a view of convex and concave surfaces of a flap of the invention, with each surface being a fragment of a torus.

FIG. 3 is a top view of a heart valve prosthesis with flaps in the closed position.

FIG. 4 is a top view of a heart valve prosthesis with flaps in the open position.

FIG. 5 is a bottom view of a heart valve prosthesis with flaps in the open position.

FIG. 6 is a view on a diametrical plane of the heart valve prosthesis with flaps in the open position.

### DETAILED DESCRIPTION OF THE PREFERRED EMBODIMENTS

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

The invention generally relates to artificial heart valve prostheses. More specifically, the invention relates to tri-leaflet mechanical heart valves.

Shown in FIG. 1 is heart valve prosthesis 10, which includes annulus 12, housing closing element 14. Annulus 12 has a central axis, annulus axis Z, and an outer surface 16 with an outer diameter referred to herein as annulus diameter D. Preferably, at least a portion of surface 16 is cylindrically shaped.

Flanges 18 and 20 are positioned, respectively, at top region 22 and bottom region 24 of annulus 12. The top region is that in which the closing element opens. Preferably, flanges 18 and 20 are the same.

Annulus 12 is fabricated from a suitable material, often titanium or titanium-based. In one example, the annulus, also referred to as “casing”, is doped with carbon ions.

For example, the annulus can be manufactured using an ion implantation apparatus which includes a vacuum chamber designed for processing multiple work pieces, e.g., casings. The vacuum chamber can include a plurality of rotatable holders, mounted on a rotatable disk in an arrangement in which only one holder is on a diametrical direction.

The ion implantation process can include propagating an ion beam along an ion beam pathway, rotating a disk having a plurality of holders to bring a casing supported by a holder in the ion beam pathway and rotating the holder to expose different surfaces of the casing to the ion beam. Preferably, not more than one holder is on a diametrical direction.

During the ion implantation process, the annulus can be moved by a method which includes rotating a disk around a disk rotational axis to impart a first movement component to the annulus being supported by a holder mounted on the disk; and spinning the holder around a holder rotational axis to impart a second movement component to the annulus. The annulus is tilted with respect to the disk rotational axis, to the holder rotational axis and with respect to the pathway of the ion beam.

Closing element 14 includes three flaps 26, shown in FIG. 1 in the closed position. Each of the flaps, also referred to as “leaflets”, is attached to annulus 12 through two hinge mechanisms 28. Hinge mechanisms 28 can use, for example, protrusions, ridges, ledges, lugs, pivots, bearings or other hinge devices known in the art. Hemispherical pivots are preferred.

In one example, flaps 26 are attached to annulus 12 by an arrangement such as described in U.S. Pat. No. 6,569,197 B2, issued to Samkov et al. on May 27, 2003, the teachings of which are incorporated herein by reference in their entirety. For instance, recesses can be provided to engage bearings, with the recesses preferably having a lateral cylindrical surface and a concave, e.g., spherical bottom.

As disclosed in U.S. Pat. No. 6,569,197 B2, recesses also can be provided in the form of a triad of blind holes being in communication via lateral surfaces. The triad preferably can have a central hole larger than the two side holes. The central hole is intended for mounting a flap bearing while the two side holes allow for blood evacuation from the hinge area. In one example, the smaller holes have a lateral cylindrical surface with a smooth concave bottom. As a variant, they can be semispherical.

Flaps 26 can be fabricated from a titanium or graphite substrate, coated with pyrolytic carbon, also known as pyrocarbon, or can be made entirely from pyrocarbon. Other suitable materials also can be employed. In a specific example, flaps 26 are manufactured as disclosed in U.S. Provisional Patent Application 60/675,982, filed on Apr. 29, 2005 and in PCT Patent Application filed concurrently herewith under Attorney Docket No. 105.5WO and entitled “A Method for Manufacturing a Leaflet for Heart Valve Prostheses”. The teachings of these two applications are incorporated herein by reference in their entirety.

For instance, a carbon-containing precursor, present in a gas having a flow direction, is pyrolyzed to produce a pyrocarbon deposit on a substrate. The substrate has a fixed orientation with respect to the flow direction. A leaflet is formed from the deposit wherein a conjugate axis of the leaflet is oriented along the flow direction. In many leaflet designs the conjugate axis refers to the conjugate axis of the lugs. The leaflet can be formed before, during or after separation of the substrate from the deposit. In preferred examples the substrate is made of graphite.

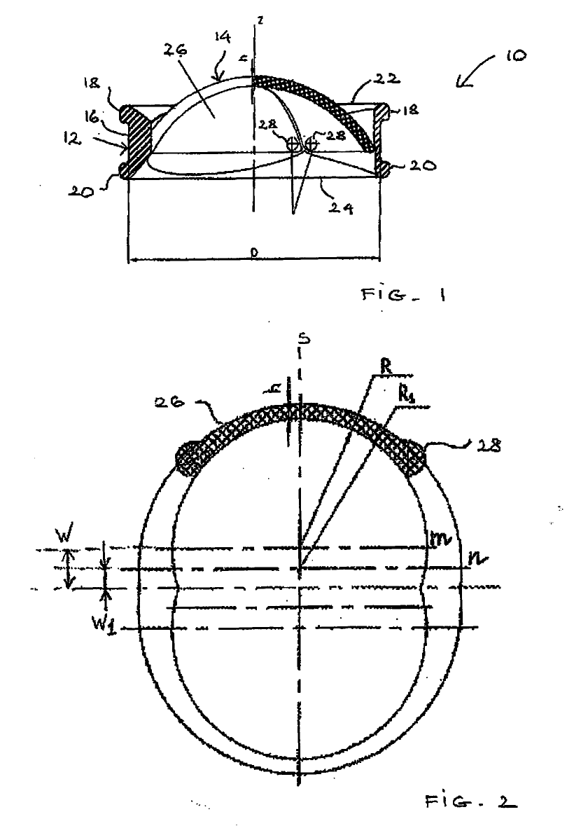

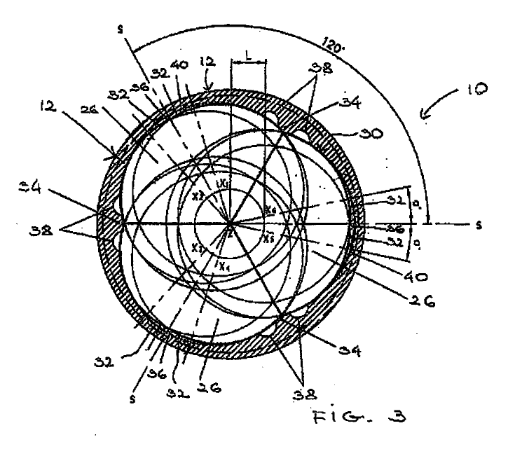

Preferably, flaps 26 are identical and each has convexo-concave surfaces, a symmetry plane S and a flap thickness h, as shown in FIG. 2. In a preferred embodiment, h is not uniform but varies from symmetry plane S to a region at or near hinge mechanism 28. Further preferred are flaps in which the value of h increases from symmetry plane S towards the regions at or near hinge mechanisms 28. In specific examples h is increased from symmetry plane S to hinge mechanisms 28 by a factor within the range of from about 1.13 to about 1.35.

In one aspect of the invention, each of convex and concave surfaces of flap 26 is a segment, also referred to as “fragment” or “part” of a toroidal surface. In geometry, a torus is a shape generated by revolving a circle about an axis coplanar with the circle. One of the most common examples of a torus has the shape of a doughnut.

In the embodiment shown in FIG. 2, the concave surface of flap 26 is generated by revolving a circle of radius R about axis m, while the convex surface is generated by revolving a circle of radius R₁ about axis n. R and R₁ preferably are in the range of from about 0.3 to about 0.6 of annulus diameter D. The distance w, between axis m and the center of the circle having radius R, is in the range within from about 0.1 of R to about 0.3 of R The distance w₁, between axis n and the center of the circle having radius R₁, is in the range within from about 0.1 of R₁ to about 0.3 of R₁. Preferably, the magnitude of R is different form that of R₁.

In other examples of the invention, each of the leaflets is convexoconcave and has a uniform thickness h′. Its surface is shaped as a fragment or part of a torus generated by revolving a circle of radius R′ about an axis. R′ can have a magnitude in the range of from about 0.6 and about 0.8 of annulus diameter D and the axis preferably is located at a distance w′ in the range of from about 0.4 to about 0.6 of R′ with respect to the center of the circle having radius R′.

Specific examples of how the flaps are attached to and engage with the interior of the heart valve casing are further described below.

FIG. 3 is a top view of heart valve prosthesis 10 which includes annulus 12 and flaps 26 in the closed position. Annulus 12 has interior surface 30. At top region 22, interior surface 30 of annulus 12 is shaped by three pairs of intersecting cylindrical surfaces 32 disposed at 120 degrees (°) of one another and preferably having the same diameter. The diameter of cylindrical surfaces 32 can be in the range of from about 0.5 to about 0.7 of annulus diameter D. Cylindrical axes x (x₁-x₆) of cylindrical surfaces 32 are displaced from annulus axis Z by a distance L. Preferably L is in the range of from about ⅛ to about ¼ of annulus diameter D.

Within each pair, cylindrical surfaces 32 have axes xₐ and x_{b}, e.g., x₁ and x₂, which are displaced from one another. If an arc of a circle is described with respect to annulus axis Z, the displacement of cylindrical axis xₐ with respect to cylindrical axis x_{b} is in the range of from about 18° to about 40°.

Intersections of cylindrical surfaces 32 described above generate two types of protrusions or ridges at the top region of interior surface 30, specifically three ledges 34 and three ledges 36. Ledges 34 preferably are evenly disposed within annulus 12, as are ledges 36. More specifically, the centers of ledges 36 are disposed at 120° from one to the next, as are the centers of ledges 34. As shown in FIG. 3, each ledge 34 alternates with a ledge 36. Thus each ledge 34 is positioned between two ledges 36.

In preferred embodiments, ledges 34 are different from ledges 36. In many aspects of the invention, ledges 34 are larger than ledges 36.

Two sockets 38 for engaging hinge mechanisms 28 of flaps 26 are recessed within surface 30 at each ledge 34. Preferably sockets 38 are hemispherically shaped to receive hemispherical pivots.

Ledges 36 receive peripheral edges of flaps 26 in the closed position.

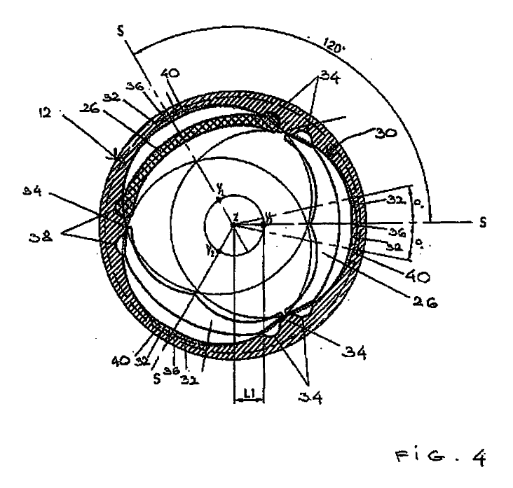

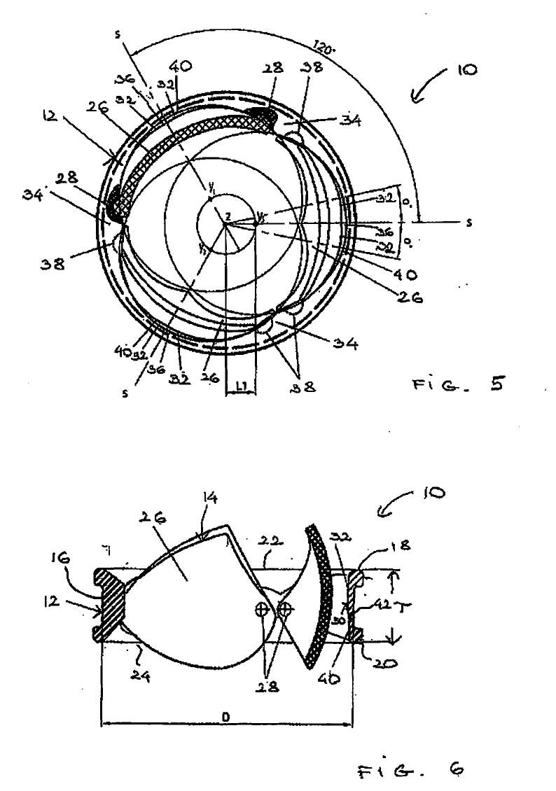

At bottom region 24, interior surface 30 of annulus 12 is shaped by three cylindrical surfaces 40 preferably having the same diameter and disposed at 120° with respect to one another, as shown in FIG. 4, which is a top view of heart valve prosthesis 10 with flaps 26 in the open position and in FIG. 5, which is a bottom view of heart valve prosthesis 10 also in the open position. Cylindrical surfaces 40 can have a diameter within the range of from about 0.6 to about 0.8 of annulus diameter D. Cylindrical axes y, specifically, y₁-y₃, of cylindrical surfaces 40 are displaced by a distance L1 from annulus axis Z. Preferably, L1 is in the range of from about 1/10 to about ⅕ of annulus diameter D.

The segments most recessed into interior surface 30, formed by cylindrical surfaces 40 at bottom region 24, are opposite, preferably diametrically opposite, to ledges 34 at top region 22. Ledges 36 at top region 22 preferably are essentially lined up with the most recessed segments formed by cylindrical surfaces 40 at bottom region 24.

One example of a transition in the shape of interior surface 30 from top region 22 to bottom region 24 is shown in FIG. 6. Shown in FIG. 6 is heart valve prosthesis 10, including annulus 12 and flaps 26, shown in the open position. Within annulus height T, interior surface 30 includes an upper portion shaped by pairs of cylindrical surfaces 32, a lower portion shaped by cylindrical surfaces 40 and a transition region 42. Transition region 42 can be stepwise or tapered and preferably is located within the lower half with respect to height T. Other placements can be employed for transition region 42.

In the manufacturing process, the dimensions of heart valve prosthesis 10 are sized for its intended use. For example, annulus diameter D can be in the range of from about 17 millimeters (mm) to about 23 mm. Annulus height T can be in the range of from about 0.2 D to about 0.4 D. The approximate wall thickness of annulus 12 can be in the range of from about 3 mm to about 5 mm; at recessed regions, annulus 12 can be as thin as from about 2.5 mm to about 3.5 mm.

During operation, flaps 26 act as occluders for the opening and closing of the valve, which can operate from 0 to about 200 times a minute. In one cycle, flaps 26, initially in a closed position, open by turning about an imaginary axis that connects opposite hinge mechanisms 28, before their installation and in an equilibrium position. The blood flow thus is direct. At the end of the direct flow, flaps 26 turn back to their closed position. Specifically, in the closed position, peripheral edges of flaps 26 engage or make contact with ledges 36. Ledges 34 and 36 together with flaps 26 provide independent flow channels for washing the region of hinge mechanisms 28.

In further embodiments, the heart valve prosthesis has an annulus with an interior surface that is shaped by the intersection of three cylindrical surfaces evenly disposed with respect to the interior circumference of the annulus and having the same diameter. The cylindrical axes of the three surfaces are shifted with respect to annulus axis Z by a length, L′, preferably within the range of from about ⅙ to about ¼ of the diameter D. Three evenly spaced ledges are formed at the intersection of the cylindrical surfaces. Each ledge is provided with recesses for engaging leaflet hinge mechanisms. For hemisherically shaped pivots, the recesses preferably are hemispherically shaped sockets.

The artificial heart valve of the invention can further include means for attachment at the implantation site, such as, for instance, a sewing ring made from woven materials. Other arrangements for attachment also can be used.

While this invention has been particularly shown and described with references to preferred embodiments thereof, it will be understood by those skilled in the art that various changes in form and details may be made therein without departing from the scope of the invention encompassed by the appended claims.

What is claimed is:

1. A heart valve prosthesis comprising three leaflets attached to an annulus, each leaflet having convexo-concave surfaces and a non-uniform thickness.

2. The heart valve prosthesis of claim 1, wherein said thickness increases from a leaflet symmetry plane to a hinge mechanism.

3. The heart valve prosthesis of claim 1, wherein each of the convexo-concave surfaces is a segment of a torus.

4. The heart valve prosthesis of claim 1, wherein an interior surface of the annulus has at least three evenly spaced ledges.

5. The heart valve prosthesis of claim 4, wherein each ledge has two sockets for attaching the leaflets.

6. The heart valve prosthesis of claim 1, wherein an interior surface of the annulus has a top region shaped by intersecting three pairs of cylindrical surfaces.

7. The heart valve prosthesis of claim 6, wherein the intersections of the three pairs of cylindrical surfaces form two sets of ledges.

8. The heart valve prosthesis of claim 6, wherein the interior surface of the annulus has a bottom region shaped by intersecting three cylindrical surfaces.

9. The heart valve prosthesis of claim 1, wherein the leaflets are made of pyrocarbon.

10. The heart valve prosthesis of claim 1, wherein the annulus is made of titanium-based material doped with carbon ions.

11. A heart valve prosthesis, comprising:
a. a closing element having three flaps;
b. an annulus for housing the closing element, the annulus having an interior surface with a top region;
c. a first set of ledges at the top region, evenly disposed about the interior surface; and
b. a second set of ledges at the top region, evenly disposed about the interior surface, each ledge in the second set alternating with a ledge in the first set.

12. The heart valve prosthesis of claim 11, wherein the first and second sets of ledges are formed by intersecting three pairs of cylindrical surfaces.

13. The heart valve prosthesis of claim 11, wherein a bottom region of the interior surface is formed by intersecting three cylindrical surfaces.

14. The heart valve prosthesis of claim 11, wherein the interior surface has two recesses at each of the first set ledges for engaging hinge mechanisms in the three flaps.

15. The heart valve prosthesis of claim 11, wherein ledges in the second set engage peripheral edges of the three flaps when in the closed position.

16. The heart valve prosthesis of claim 11, wherein the flaps have a thickness which is uniform.

17. The heart valve prosthesis of claim 11, wherein the flaps have a non-uniform thickness.

18. The heart valve prosthesis of claim 11, wherein the flaps are made of pyrocarbon.

19. The heart valve prosthesis of claim 11, wherein the annulus is made of a titanium-based material doped with carbon ions.

20. A leaflet shaped for a tri-leaflet artificial heart valve, having a concave surface, a convex surface and a non-uniform thickness.

21. The leaflet of claim 20, further comprising two hinge mechanisms for engaging with a casing.

22. The leaflet of claim 20, wherein the concave surface is generated by revolving a circle of radius R about an axis m displaced with respect to the center of the circle having radius R and wherein the convex surface is generated by revolving a circle of radius R₁ about an axis n, displaced with respect to the center of the circle having radius R₁.

23. The leaflet of claim 20, wherein the leaflet has a peripheral edge shaped for engaging a ledge on an interior surface of a casing.

24. A heart valve prosthesis, comprising:
a. a closing element having three flaps;
b. an annulus for housing the closing element, the annulus having an annulus axis and an interior surface;
c. three ledges formed at the interior surface by three intersecting cylindrical surfaces evenly disposed within the annulus, each cylindrical surface having a cylindrical axis displaced from the annulus axis; and
d. two sockets at each ledge for attaching said flaps to the interior surface.
